(19) United States
(12) Patent Application Publication
(10) Pub. No.: US 2012/0269948 A1
(43) Pub. Date: Oct. 25, 2012

(54) CANOLA PROTEIN PRODUCT FROM SUPERNATANT

(76) Inventors: Sarah Medina, Winnipeg (CA); Martin Schweizer, Winnipeg (CA); Brent E. Green, Warren (CA); Kevin I. Segall, Winnipeg (CA); Randy Willardsen, Roseville, CA (US); James Logie, Winnipeg (CA)

(21) Appl. No.: 13/394,858

(22) PCT Filed: Sep. 14, 2010
(86) PCT No.: PCT/CA2010/001424

§ 371 Date: Jul. 4, 2012

Related U.S. Application Data
(60) Provisional application No. 61/272,359, filed on Sep. 17, 2009.

(51) Int. Cl. C07K 14/415; C07K 1/36; A23J 3/14; C07K 1/30

(52) U.S. Cl. 426/590; 530/350; 530/418; 530/414; 530/419

(57) ABSTRACT

A novel canola protein product consisting predominantly of 2S canola protein and having improved solubility properties, has an increased proportion of 2 S canola protein and a decreased proportion of 7S canola protein, and a protein content of less than about 90 wt % (N×6.25) d.b. The novel canola protein isolate is formed by heat treatment or isoelectric precipitation of aqueous supernatant from canola protein micelle formation and precipitation, to effect precipitation of 7S protein which is sedimented and removed.

### FIELD OF THE INVENTION

The present invention relates to the production of canola protein products and their use in aqueous solution, including soft drinks and sports drinks.

### BACKGROUND TO THE INVENTION

In copending U.S. patent applications Ser. No. 11/038,086 filed Jan. 21, 2005 (US Patent Application Publication No. 2005-0181112, WO 2005/067729) and Ser. No. 12/213,500 filed Jun. 20, 2008 (US Patent Application Publication No. 2008-0299282, WO 2009/152621), assigned to the Assignee hereof and the disclosures of which are incorporated herein by reference, there is described the production of a canola protein isolate having a protein content of at least about 90 wt % (N×6.25) on a dry weight basis (d.b.), preferably at least about 100 wt %, by heat treatment or isoelectric precipitation of the supernatant, which may be partially concentrated or concentrated, from the deposition of a canola protein micellar mass to cause precipitation of canola 7S protein from the supernatant. Following removal of the precipitated canola 7S protein, the treated supernatant is dried.

The canola protein isolate so formed, enhanced in 2S protein content in comparison to the untreated supernatant, exhibits superior properties in aqueous solution to canola protein isolate derived directly from untreated supernatant. In addition to equal or greater solubility at a variety of pH values, the 2S-enriched canola protein isolate provided therein is able to provide improved clarity in solution with soft drinks and sports drinks, providing clear protein fortified beverages, including acidic beverages, such as soft drinks and sports drinks.

Canola is also known as rapeseed or oil seed rape.

### SUMMARY OF THE INVENTION

It has now been found that, if the procedures described in the aforementioned U.S. Ser. Nos. 11/038,086 and 12/213,500 are effected in such a manner that the 2S-enriched canola protein product contains less than about 90 wt % (N×6.25) d.b. and hence is not an isolate, such as at least about 60 wt % (N×6.25) d.b., the concentration at which the canola protein product is considered to be a concentrate, there is obtained a canola protein product which similarly is completely soluble over a wide range of pH values and which is able to provide clear protein fortified beverages, including acidic beverages, such as soft drinks and sports drinks.

This result is achieved herein by omitting or reducing the diafiltration step effected on the supernatant or by stopping the ultrafiltration step effected on the supernatant earlier, so that fewer contaminants are removed during these steps and hence a canola protein product is obtained with lesser purity than an isolate.

In accordance with one aspect of the present invention, there is provided a process for the preparation of a canola protein product having an increased proportion of 2S canola protein, which comprises:
(a) providing an aqueous solution of 2S and 7S proteins consisting predominantly of 2S protein,
(b) heat treating the aqueous solution to cause precipitation of 7S canola protein,
(c) removing precipitated 7S protein from the aqueous solution, and
(d) recovering a canola protein product having a protein content of less than about 90 wt % (N×6.25) d.b. and having an increased proportion of 2S canola protein.

In accordance with another aspect of the present invention, there is provided a process for the preparation of a canola protein product having an increased proportion of 2S canola protein, which comprises:
(a) providing an aqueous solution of 2S and 7S proteins consisting predominantly of 2S protein,
(b) isoelectrically precipitating 7S protein from the aqueous solution,
(c) removing precipitated 7S protein from the aqueous solution, and,
(d) recovering a canola protein product having a protein content of less than about 90 wt % (N×6.25) d.b. and having an increased proportion of 2S canola protein compared to the aqueous solution of 2S and 7S proteins.

The canola protein products produced herein contain at least about 85 wt % of 2S canola protein and less than about 15 wt % of 7S canola protein, preferably at least about 90 wt % of 2S canola protein and less than about 10 wt % of 7S canola protein and more preferably as great a proportion of 2S protein as is possible. As noted above, such canola protein product is obtained by heat treatment or isoelectric precipitation of supernatant, partially concentrated supernatant and concentrated supernatant, as described in more detail below. The heat treatment or isoelectric precipitation of the supernatant, partially concentrated supernatant and concentrated supernatant causes precipitation of the 7S protein, which can be removed from the treated supernatant by any convenient means, such as centrifugation or filtration. The 2S protein is not affected by the treatment and hence the treatment increases the proportion of 2S protein present by decreasing the proportion of 7S protein.

The canola protein product is soluble in aqueous solution over a wide range of pH values, generally about pH 2 to about pH 7.5, preferably about 2 to about 4, generally having solubility equal to or greater than canola protein product consisting predominantly of 2S protein and derived directly from supernatant from canola protein micelle formation and precipitation under the same experimental conditions of preparation. In addition, aqueous solutions of the canola protein product in soft drinks, including both carbonated and non-carbonated soft drinks and sports drinks, including both carbonated and non-carbonated sports energy drinks, such as those commercially-available, have a greater clarity than such aqueous solutions produced from canola protein product consisting predominantly of 2S protein and derived directly from supernatant from canola protein micelle formation and precipitation under the same conditions of preparation.

The concentration of canola protein product in the aqueous solution, including solution in soft drinks and sports drinks, may vary depending on the intended use of the solution. In general, the protein concentration may vary from about 0.1 to about 30 wt %, preferably about 1 to about 5 wt %.

The canola protein products provided herein are suitable, not only for protein fortification of acid media, but may be used in a wide variety of conventional applications of protein products, including but not limited to protein fortification of processed foods and beverages, emulsification of oils, as a body former in baked goods and foaming agent in products which entrap gases. In addition, the canola protein products may be formed into protein fibers, useful in meat analogs and may be used as an egg white substitute or extender in food products where egg white is used as a binder. The canola protein products may also be used in nutritional supplements. Other uses of the canola protein products are in pet foods, animal feed and in industrial and cosmetic applications and in personal care products.

### GENERAL DESCRIPTION OF INVENTION

Accordingly, the present invention includes aqueous solutions of the canola protein product provided herein, including not only those mentioned above, but also other beverages, such as juices, alcoholic beverages, coffee-based beverages and dairy-based beverages.

The initial step of the process of providing canola protein products involves solubilizing proteinaceous material from canola oil seed meal. The proteinaceous material recovered from canola seed meal may be the protein naturally occurring in canola seed or the proteinaceous material may be a protein modified by genetic manipulation but possessing characteristic hydrophobic and polar properties of the natural protein. The canola meal may be any canola meal resulting from the removal of canola oil from canola oil seed with varying levels of non-denatured protein, resulting, for example, from hot hexane extraction or cold oil extrusion methods. The removal of canola oil from canola oil seed usually is effected as a separate operation from the canola protein product recovery procedure described herein.

Protein solubilization is effected most efficiently by using a food grade salt solution since the presence of the salt enhances the removal of soluble protein from the oil seed meal. Where the canola protein isolate is intended for non-food uses, non-food-grade chemicals may be used. The salt usually is sodium chloride, although other salts, such as, potassium chloride, may be used. The salt solution has an ionic strength of at least about 0.05, preferably at least about 0.10, to enable solubilization of significant quantities of protein to be effected. As the ionic strength of the salt solution increases, the degree of solubilization of protein in the oil seed meal initially increases until a maximum value is achieved. Any subsequent increase in ionic strength does not increase the total protein solubilized. The ionic strength of the food grade salt solution which causes maximum protein solubilization varies depending on the salt concerned and the oil seed meal chosen.

In view of the greater degree of dilution required for protein precipitation with increasing ionic strengths, it is usually preferred to utilize an ionic strength value less than about 0.8, and more preferably a value of about 0.1 to about 0.15.

In a batch process, the salt solubilization of the protein is effected at a temperature of from about 5° C. to about 75° C., preferably accompanied by agitation to decrease the solubilization time, which is usually about 10 to about 60 minutes. It is preferred to effect the solubilization to extract substantially as much protein from the oil seed meal as is practicable, so as to provide an overall high product yield.

The lower temperature limit of about 5° C. is chosen since solubilization is impractically slow below this temperature while the upper preferred temperature limit of about 75° C. is chosen due to the denaturation temperature of some of the present proteins.

In a continuous process, the extraction of the protein from the canola oil seed meal is carried out in any manner consistent with effecting a continuous extraction of protein from the canola oil seed meal. In one embodiment, the canola oil seed meal is continuously mixed with a food grade salt solution and the mixture is conveyed through a pipe or conduit having a length and at a flow rate for a residence time sufficient to effect the desired extraction in accordance with the parameters described herein. In such continuous procedure, the salt solubilization step is effected rapidly, in a time of up to about 10 minutes, preferably to effect solubilization to extract substantially as much protein from the canola oil seed meal as is practicable. The solubilization in the continuous procedure is effected at temperatures between about 10° C. and about 75° C., preferably between about 15° C. and about 35° C.

The aqueous food grade salt solution generally has a pH of about 5 to about 6.8, preferably about 5.3 to about 6.2, the pH of the salt solution may be adjusted to any desired value within the range of about 5 to about 6.8 for use in the extraction step by the use of any convenient acid, usually hydrochloric acid, or alkali, usually sodium hydroxide, as required.

The concentration of oil seed meal in the food grade salt solution during the solubilization step may vary widely. Typical concentration values are about 5 to about 15% w/v.

The protein extraction step with the aqueous salt solution has the additional effect of solubilizing fats which may be present in the canola meal, which then results in the fats being present in the aqueous phase.

The protein solution resulting from the extraction step generally has a protein concentration of about 5 to about 40 g/L, preferably about 10 to about 30 g/L.

The aqueous salt solution may contain an antioxidant. The antioxidant may be any convenient antioxidant, such as sodium sulfite or ascorbic acid. The quantity of antioxidant employed may vary from about 0.01 to about 1 wt % of the solution, preferably about 0.05 wt %. The antioxidant serves to inhibit oxidation of phenolics in the protein solution.

The aqueous phase resulting from the extraction step then may be separated from the residual canola meal, in any convenient manner, such as by employing a decanter centrifuge, followed by disc centrifugation and/or filtration to remove residual meal. The separated residual meal may be dried for disposal.

The colour of the canola protein product recovered from the canola protein solution can be improved in terms of light colour and less intense yellow by the mixing of powdered activated carbon or other pigment adsorbing agent with the separated aqueous protein solution and subsequently removing the adsorbent, conveniently by filtration, to provide a protein solution. Diafiltration of the canola protein solution also may be used for pigment removal.

Such pigment removal step may be carried out under any convenient conditions, generally at the ambient temperature of the separated aqueous protein solution, employing any suitable pigment adsorbing agent. For powdered activated carbon, an amount of about 0.025% to about 5% w/v, preferably about 0.05% to about 2% w/v, is employed.

Where the canola seed meal contains significant quantities of fat, as described in U.S. Pat. Nos. 5,844,086 and 6,005,076, assigned to the assignee hereof and the disclosures of which are incorporated herein by reference, then the defatting steps described therein or any other convenient defatting procedure, may be effected on the separated aqueous protein solution and on the concentrated aqueous protein solution discussed below. When the colour improvement step is carried out, such step may be effected after the first defatting step.

As an alternative to extracting the oil seed meal with an aqueous salt solution, such extraction may be made using water alone, although the utilization of water alone tends to extract less protein from the oil seed meal than the aqueous salt solution. Where such alternative is employed, then the salt, in the concentrations discussed above, may be added to the protein solution after separation from the residual oil seed meal in order to maintain the protein in solution during the concentration step described below. When a first fat removal step is carried out, the salt generally is added after completion of such operations.

Another alternative procedure is to extract the oil seed meal with the food grade salt solution at a relatively high pH value above about 6.8, generally up to about 9.9. The pH of the food grade salt solution may be adjusted to the desired alkaline value by the use of any convenient food-grade alkali, such as aqueous sodium hydroxide solution. Alternatively, the oil seed meal may be extracted with the salt solution at a relatively low pH below about pH 5, generally down to about pH 3. Where such alternative is employed, the aqueous phase resulting from the oil seed meal extraction step then is separated from the residual canola meal, in any convenient manner, such as by employing decanter centrifugation, followed by disc centrifugation and/or filtration to remove residual meal. The separated residual meal may be dried for disposal.

The aqueous protein solution resulting from the high or low pH extraction step then is pH adjusted to the range of about 5 to about 6.8, preferably about 5.3 to about 6.2, as discussed above, prior to further processing as discussed below. Such pH adjustment may be effected using any convenient acid, such as hydrochloric acid, or alkali, such as sodium hydroxide, as appropriate.

The aqueous protein solution is concentrated to increase the protein concentration thereof while maintaining the ionic strength thereof substantially constant. Such concentration generally is effected to provide a concentrated protein solution having a protein concentration of at least about 50 g/L, preferably at least about 200 g/L, more preferably at least about 250 g/L.

The concentration step may be effected in any convenient manner consistent with batch or continuous operation, such as by employing any convenient selective membrane technique, such as ultrafiltration or diafiltration, using membranes, such as hollow-fibre membranes or spiral-wound membranes, with a suitable molecular weight cut-off, such as about 3,000 to about 100,000 daltons, preferably about 5,000 to about 10,000 daltons, having regard to differing membrane materials and configurations, and, for continuous operation, dimensioned to permit the desired degree of concentration as the aqueous protein solution passes through the membranes.

As is well known, ultrafiltration and similar selective membrane techniques permit low molecular weight species to pass through the membrane while preventing higher molecular weight species from so doing. The low molecular weight species include not only the ionic species of the food grade salt but also low molecular weight materials extracted from the source material, such as, carbohydrates, pigments and anti-nutritional factors, as well as any low molecular weight forms of the protein. The molecular weight cut-off of the membrane is usually chosen to ensure retention of a significant proportion of the protein in the solution, while permitting contaminants to pass through having regard to the different membrane materials and configurations.

The concentrated protein solution then may be subjected to a diafiltration step using an aqueous salt solution of the same molarity and pH as the extraction solution. Such diafiltration may be effected using from about 2 to about 20 volumes of diafiltration solution, preferably about 5 to about 10 volumes of diafiltration solution. In the diafiltration operation, further quantities of contaminants are removed from the aqueous protein solution by passage through the membrane with the permeate. The diafiltration operation may be effected until no significant further quantities of contaminants and visible colour are present in the permeate. Such diafiltration may be effected using the same membrane as for the concentration step. However, if desired, the diafiltration step may be effected using a separate membrane with a different molecular weight cut-off, such as a membrane having a molecular weight cut-off in the range of about 3,000 to about 100,000 daltons, preferably about 5,000 to about 10,000 daltons, having regard to different membrane materials and configuration.

An antioxidant may be present in the diafiltration medium during at least part of the diafiltration step. The antioxidant may be any convenient antioxidant, such as sodium sulfite or ascorbic acid. The quantity of antioxidant employed in the diafiltration medium depends on the materials employed and may vary from about 0.01 to about 1 wt %, preferably about 0.05 wt %. The antioxidant serves to inhibit oxidation of phenolics present in the concentrated canola protein isolate solution.

The concentration step and the diafiltration step may be effected at any convenient temperature, generally about 20° to about 60° C., preferably about 20 to about 30° C., and for the period of time to effect the desired degree of concentration. The temperature and other conditions used to some degree depend upon the membrane equipment used to effect the concentration and the desired protein concentration of the solution.

The concentrated and optionally diafiltered protein solution may be subjected to a further defatting operation, if required, as described in U.S. Pat. Nos. 5,844,086 and 6,005,076.

The concentrated and optionally diafiltered protein solution may be subjected to a colour removal operation as an alternative to the colour removal operation described above. Powdered activated carbon may be used herein as well as granulated activated carbon (GAC). Another material which may be used as a colour adsorbing agent is polyvinyl pyrrolidone.

The colour adsorbing agent treatment step may be carried out under any convenient conditions, generally at the ambient temperature of the concentrated and optionally diafiltered canola protein solution. For powdered activated carbon, an amount of about 0.025% to about 5% w/v, preferably about 0.05% to about 2% w/v, may be used. Where polyvinylpyrrolidone is used as the colour adsorbing agent, an amount of about 0.5% to about 5% w/v, preferably about 2% to about 3% w/v, may be used. The colour adsorbing agent may be removed from the canola protein solution by any convenient means, such as by filtration.

The concentrated and optionally diafiltered protein solution resulting from the optional colour removal step may be subjected to pasteurization to reduce the microbial load. Such pasteurization may be effected under any desired pasteurization conditions.

Generally, the concentrated and optionally diafiltered protein solution is heated to a temperature of about 55° to about 70° C., preferably about 60° to about 65° C., for about 10 to about 15 minutes, preferably about 10 minutes. The pasteurized concentrated protein solution then may be cooled for further processing as described below, preferably to a temperature of about 25° to about 40° C.

Depending on the temperature employed in the concentration step and optional diafiltration step and whether or not a pasteurization step is effected, the concentrated protein solution may be warmed to a temperature of at least about 20°, and up to about 60° C., preferably about 25° to about 40° C., to decrease the viscosity of the concentrated protein solution to facilitate performance of the subsequent dilution step and micelle formation. The concentrated protein solution should not be heated beyond a temperature above which micelle formation does not occur on dilution by chilled water.

The concentrated protein solution resulting from the concentration step, and optional diafiltration step, optional colour removal step, optional pasteurization step and optional defatting step, then is diluted to effect micelle formation by mixing the concentrated protein solution with chilled water having the volume required to achieve the degree of dilution desired. Depending on the proportion of canola protein desired to be obtained by the micelle route and the proportion from the supernatant, the degree of dilution of the concentrated protein solution may be varied. With lower dilution levels, in general, a greater proportion of the canola protein remains in the aqueous phase.

When it is desired to provide the greatest proportion of the protein by the micelle route, the concentrated protein solution is diluted by about 5 fold to about 25 fold, preferably by about 10 fold to about 20 fold.

The chilled water with which the concentrated protein solution is mixed has a temperature of less than about 15° C., generally about 1° to about 15° C., preferably less than about 10° C., since improved yields of protein isolate in the form of protein micellar mass are obtained with these colder temperatures at the dilution factors used.

In a batch operation, the batch of concentrated protein solution is added to a static body of chilled water having the desired volume, as discussed above. The dilution of the concentrated protein solution and consequential decrease in ionic strength causes the formation of a cloud-like mass of highly associated protein molecules in the form of discrete protein droplets in micellar form. In the batch procedure, the protein micelles are allowed to settle in the body of chilled water to form an aggregated, coalesced, dense, amorphous sticky gluten-like protein micellar mass (PMM). The settling may be assisted, such as by centrifugation. Such induced settling decreases the liquid content of the protein micellar mass, thereby decreasing the moisture content generally from about 70% by weight to about 95% by weight to a value of generally about 50% by weight to about 80% by weight of the total micellar mass. Decreasing the moisture content of the micellar mass in this way also decreases the occluded salt content of the micellar mass, and hence the salt content of dried isolate.

Alternatively, the dilution operation may be carried out continuously by continuously passing the concentrated protein solution to one inlet of a T-shaped pipe, while the diluting water is fed to the other inlet of the T-shaped pipe, permitting mixing in the pipe. The diluting water is fed into the T-shaped pipe at a rate sufficient to achieve the desired degree of dilution of the concentrated protein solution.

The mixing of the concentrated protein solution and the diluting water in the pipe initiates the formation of protein micelles and the mixture is continuously fed from the outlet from the T-shaped pipe into a settling vessel, from which, when full, supernatant is permitted to overflow. The mixture preferably is fed into the body of liquid in the settling vessel in a manner which minimizes turbulence within the body of liquid.

In the continuous procedure, the protein micelles are allowed to settle in the settling vessel to form an aggregated, coalesced, dense, amorphous, sticky, gluten-like protein micellar mass (PMM) and the procedure is continued until a desired quantity of the PMM has accumulated in the bottom of the settling vessel, whereupon the accumulated PMM is removed from the settling vessel. In lieu of settling by sedimentation, the PMM may be separated continuously by centrifugation.

The combination of process parameters of concentrating of the protein solution to a preferred protein content of at least about 200 g/L and the use of a dilution factor of about 10 to about 20, result in higher yields, often significantly higher yields, in terms of recovery of protein in the form of protein micellar mass from the original meal extract, and much purer isolates in terms of protein content than achieved using any of the known prior art protein isolate forming procedures discussed in the aforementioned US patents.

By the utilization of a continuous process for the recovery of canola protein isolate as compared to the batch process, the initial protein extraction step can be significantly reduced in time for the same level of protein extraction and significantly higher temperatures can be employed in the extraction step. In addition, in a continuous operation, there is less chance of contamination than in a batch procedure, leading to higher product quality and the process can be carried out in more compact equipment.

The settled canola protein isolate is separated from the residual aqueous phase or supernatant, such as by decantation of the residual aqueous phase from the settled mass or by centrifugation. The PMM may be used in the wet form or may be dried, by any convenient technique, such as spray drying or freeze drying, to a dry form. The dry PMM has a high protein content, in excess of about 90 wt % protein, preferably at least about 100 wt % protein (calculated as N×6.25), and is substantially undenatured (as determined by differential scanning calorimetry). The dry PMM isolated from fatty oil seed meal also has a low residual fat content, when the procedures of U.S. Pat. Nos. 5,844,086 and 6,005,076 are employed as necessary, which may be below about 1 wt %.

As described in U.S. Pat. No. 7,662,922 (WO 03/088760), assigned to the assignee hereof and the disclosures of which are incorporated herein by reference, the PMM consists predominantly of a 7S canola protein having a protein component content of about 60 to 98 wt % of 7S protein, about 1 to about 15 wt % of 12S protein and 0 to about 25 wt % of 2S protein.

The supernatant from the PMM formation and settling step contains significant amounts of canola protein, not precipitated in the dilution step, and is processed to recover canola protein product therefrom. As described in the aforementioned U.S. Pat. No. 7,662,922, the canola protein product derived from the supernatant consists predominantly of 2S canola protein having a protein component content of about 60 to about 95 wt % of 2S protein, about 5 to about 40 wt % of a 7S protein and 0 to about 5 wt % of 12S protein.

The supernatant is concentrated to increase the protein concentration thereof. Such concentration is effected using any convenient selective membrane technique, such as ultrafiltration, using membranes with a suitable molecular weight cut-off permitting low molecular weight species, including the salt and other non-proteinaceous low molecular weight materials extracted from the protein source material, to pass through the membrane, while retaining canola protein in the solution. Ultrafiltration membranes having a molecular weight cut-off of about 3,000 to 100,000 daltons, preferably about 5,000 to about 10,000 daltons, having regard to differing membrane materials and configuration, may be used. Concentration of the supernatant in this way also reduces the volume of liquid required to be dried to recover the protein. The supernatant generally is concentrated to a protein concentration of at least about 50 g/L, preferably about 100 to about 300 g/L, more preferably about 200 to about 300 g/L, prior to drying. Such concentration operation may be carried out in a batch mode or in a continuous operation, as described above for the protein solution concentration step.

The concentrated supernatant then may be subjected to a diafiltration step using water, saline or acidified water. Such diafiltration may be effected using from about 2 to about 20 volumes of diafiltration solution, preferably about 5 to about 10 volumes of diafiltration solution. In the diafiltration operation, further quantities of contaminants are removed from the aqueous supernatant by passage through the membrane with the permeate. The diafiltration operation may be effected until no significant further quantities of contaminants and visible colour are present in the permeate. Such diafiltration may be effected using the same membrane as for the concentration step. However, if desired, the diafiltration may be effected using a separate membrane, such as a membrane having a molecular weight cut-off in the range of about 3,000 to about 100,000 daltons, preferably about 5,000 to about 10,000 daltons, having regard to different membrane materials and configuration.

To produce a canola protein product containing less than 90 wt % protein (N×6.25) d.b., the above-described concentration and/or diafiltration steps effected on the supernatant are manipulated to remove fewer contaminants from the supernatant so that the recovered canola protein product has a protein content of less than 90 wt % protein, such as at least about 60 wt % protein (N×6.25) d.b., such as by omitting or reducing the diafiltration step and/or by stopping the ultrafiltration step earlier.

An antioxidant may be present in the diafiltration medium during at least part of the diafiltration step. The antioxidant may be any convenient antioxidant, such as sodium sulfite or ascorbic acid. The quantity of antioxidant employed in the diafiltration medium depends on the materials employed and may vary from about 0.01 to about 1 wt %, preferably about 0.05 wt %. The antioxidant serves to inhibit oxidation of phenolics present in the concentrated canola protein isolate solution.

The concentrated and optionally diafiltered protein solution may be subjected to a colour removal operation. Powdered activated carbon may be used herein as well as granulated activated carbon (GAC). Another material which may be used as a colour adsorbing agent is polyvinyl pyrrolidone.

The colour adsorbing agent treatment step may be carried out under any convenient conditions, generally at the ambient temperature of the canola protein solution. For powdered activated carbon, an amount of about 0.025% to about 5% w/v, preferably about 0.05% to about 2% w/v, may be used. Where polyvinylpyrrolidone is used as the colour adsorbing agent, an amount of about 0.5% to about 5% w/v, preferably about 2% to about 3% w/v, may be used. The colour adsorbing agent may be removed from the canola protein solution by any convenient means, such as by filtration.

In accordance with one aspect of the present invention, the concentrated and optionally diafiltered supernatant, following the optional colour removal operation, is heat treated or subjected to isoelectric precipitation to decrease the quantity of the 7S protein present in the solution by removal of the resulting precipitated 7S protein and thereby increasing the proportion of 2S protein in the canola protein present in the concentrated supernatant.

Such heat treatment may be effected using a temperature and time profile sufficient to decrease the proportion of 7S present in the concentrated supernatant, preferably to reduce the proportion of 7S protein by a significant extent. In general, the 7S protein content of the supernatant is reduced by at least about 50 wt %, preferably at least about 75 wt % by the heat treatment. In general, the heat treatment may be effected at a temperature of about 70° to about 120° C., preferably about 75° to about 105° C., for about 1 second to about 30 minutes, preferably about 5 to about 15 minutes.

The concentrated, heat-treated supernatant may be acidified, prior to drying, to a pH corresponding to the intended use of the dried product. Generally a pH down to about 2 to about 5, preferably about 2.5 to about 4.

The concentrated heat-treated supernatant, after removal of the precipitated 7S protein, may be dried by any convenient technique, such as spray drying or freeze drying, to a dry form to provide a canola protein product in accordance with the present invention. Such canola protein product has a protein content of less than about 90 wt %, preferably at least about 70 wt % protein, (calculated as N×6.25) on a dry weight basis and is expected to be substantially undenatured.

Such canola protein product contains a high proportion of 2S protein, preferably at least 90 wt % and most preferably at least about 95 wt %, of the canola protein in the product. There is also a proportion of 7S protein in the product.

Alternatively, the heat treatment of the supernatant to precipitate 7S protein may be effected on the supernatant prior to the concentration and diafiltration steps mentioned above. Following removal of the deposited 7S protein, the supernatant then is concentrated, optionally diafiltered, optionally submitted to a colour removal operation, and dried to provide the canola protein product according to the invention.

As a further alternative, the supernatant first may be partially concentrated to any convenient level. The partially concentrated supernatant then is subjected to the heat treatment to precipitate 7S protein. Following removal of the precipitated 7S protein, the supernatant is further concentrated, generally to a concentration of about 50 to about 300 g/L, preferably about 200 to about 300 g/L, optionally diafiltered, optionally submitted to a colour removal operation, and dried to provide the canola protein product according to the invention.

Precipitated 7S protein is removed from the supernatant, partially concentrated supernatant or concentrated supernatant by any convenient means, such as by centrifugation or filtration or a combination thereof.

Following removal of precipitated 7S protein, the heat treated supernatant or partially concentrated, heat treated supernatant may be acidified at any point during or after concentration or diafiltration, as discussed above, prior to drying to recover the canola protein product. Such acidification may be effected to a pH corresponding to the intended use of the dried isolate, generally a pH down to about 2 to about 5, preferably about 2.5 to about 4, for use in acid beverages.

In another embodiment of the invention, the supernatant from the micelle formation and precipitation is subjected to isoelectric precipitation to form the novel canola protein product of the invention. The supernatant may be first concentrated or partially concentrated, as discussed above with respect to the heat treatment, prior to the isoelectric precipitation.

In such isoelectric precipitation procedure, a salt, usually sodium chloride, although other salts such as potassium chloride may be used, first is added to the supernatant, partially concentrated supernatant or concentrated supernatant to provide a salinated solution having a conductivity of at least about 0.3 mS, preferably about 10 to about 20 mS.

The pH of the salinated supernatant is adjusted to a value to cause isoelectric precipitation of 7S protein, generally to a pH of about 2.0 to about 4.0, preferably about 3.0 to about 3.5. The isoelectric precipitation of the 7S protein may be effected over a wide temperature range, generally from about 5° C. to about 70° C., preferably about 10° C. to about 40° C. The precipitated 7S protein is removed from the isoelectrically precipitated supernatant by any convenient means, such as by centrifugation or filtration or a combination thereof.

The isoelectrically precipitated supernatant, if not already concentrated, then is concentrated as discussed above with respect to the heat treatment step and diafiltered to remove the salt, prior to drying the concentrated and diafiltered supernatant to form the canola protein product of the invention. The concentrated and diafiltered supernatant may be filtered to remove residual particulates and subjected to an optional colour removal step, as discussed above, prior to drying by any convenient technique, such as by spray drying or freeze drying, to a dry form to provide a canola protein product according to the present invention having less than about 90 wt % protein (N×6.25) d.b.

The canola protein product produced herein is soluble in an acidic aqueous environment, making the product ideal for incorporation into beverages, both carbonated and uncarbonated, to provide protein fortification thereto. Such beverages have a wide range of acidic pH values, ranging from about 2.5 to about 5. The canola protein product provided herein may be added to such beverages in any convenient quantity to provide protein fortification to such beverages, for example, at least about 5 g of the canola protein product per serving. The added canola protein product dissolves in the beverage and the opacity of the beverage is not increased by thermal processing. The canola protein product may be blended with dried beverage prior to reconstitution of the beverage by dissolution in water. In some cases, modification to the normal formulation of the beverage to tolerate the composition of the invention may be necessary where components present in the beverage may adversely effect to ability of the composition of the invention to remain dissolved in the beverage.

### EXAMPLES

### Example 1

This Example illustrates the production of a canola protein product of less than 90 wt % protein, dry weight, in accordance with one embodiment of the invention.

‘a’ kg of canola meal was added to ‘b’ L of ‘c’ M NaCl solution at ambient temperature and agitated for 30 minutes to provide an aqueous protein solution. The residual canola meal was removed and the resulting protein solution was partially clarified by centrifugation to produce ‘d’ L of partially clarified protein solution having a protein content of ‘e’ % by weight. The partially clarified protein solution was then filtered to further clarify resulting in a solution of volume ‘f’ having a protein content of ‘g’ by weight.

A ‘h’ L aliquot of the protein extract solution was reduced in volume to ‘i’ L by concentration on a polyethersulfone (PES) membrane having a molecular weight cut-off of ‘j’ daltons. This retentate was then pasteurized at 60° C. for 1 minute. The resulting pasteurized concentrated protein solution had a protein content of ‘k’ % by weight.

The concentrated solution at ‘l’ ° C. was diluted ‘m’ into cold RO water having a temperature of ‘n’ ° C. A white cloud formed immediately and was allowed to settle. The upper diluting water was removed and the precipitated, viscous, sticky mass (PMM) was recovered by centrifugation in a yield of ‘o’ wt % of the filtered protein solution and dried. The dried PMM protein was found to have a protein content of ‘p’ wt % (N×6.25) d.b. The product was given a designation ‘q’ C300.

The heat treatment described above was then carried out on the supernatant.

‘r’ L supernatant was heated to 80° C. for 10 minutes and then centrifuged to remove precipitated protein. The centrifuged heat-treated supernatant was reduced in volume to ‘s’ L by ultrafiltration using a polyethersulfone (PES) membrane having a molecular weight cut-off of ‘t’ Daltons and the concentrate was then diafiltered on the same membrane with ‘u’ L of water adjusted to a conductivity of 1 mS with sodium chloride. The diafiltered concentrate contained ‘v’ % protein by weight. With the additional protein recovered from the supernatant, the overall protein recovery of the filtered protein solution was ‘w’ wt %. A ‘x’ L portion of the concentrate was adjusted to pH 3 with HCl and subjected to a colour reduction step by passing it through a ‘y’ L bed volume of granular activated carbon at a rate of ‘z’ BV/hr at pH 3. The ‘aa’ L of GAC treated solution having reduced colour and a protein content of ‘ab’ % by weight was then spray dried, given the designation ‘s’ C200HSC and had a protein content of ‘ac’ wt % (N×6.25) d.b. The parameters ‘a’ to ‘ac’ for one run are set forth in the following Table I below.

### Example 2

This Example illustrates the production of a novel canola protein product of less than 90% protein by weight in accordance with another embodiment of the invention.

‘a’ kg of canola meal was added to ‘b’ L of ‘c’ M NaCl solution at ambient temperature and agitated for 30 minutes to provide an aqueous protein solution. The residual canola meal was removed and the resulting protein solution was partially clarified by centrifugation to produce ‘d’ L of partially clarified protein solution having a protein content of ‘e’ % by weight. The partially clarified protein solution was then filtered to further clarify, resulting in a solution of volume ‘f’ having a protein content of ‘g’ by weight.

A ‘h’ L aliquot of the protein extract solution was reduced in volume to ‘i’ L by concentration on a polyvinylidene fluoride (PVDF) membrane having a molecular weight cut-off of ‘j’ daltons. This retentate was then pasteurized at 60° C. for 10 minutes. The resulting pasteurized concentrated protein solution had a protein content of ‘k’ % by weight.

The concentrated solution at ‘l’ ° C. was diluted ‘m’ into cold RO water having a temperature ‘n’ ° C. A white cloud formed immediately and was allowed to settle. The upper diluting water was removed and the precipitated, viscous, sticky mass (PMM) was recovered by centrifugation in a yield of ‘o’ wt % of the filtered protein solution and spray dried. The dried PMM derived protein was found to have a protein content of ‘p’ wt % (N×6.25) d.b. The product was given a designation ‘q’ C300.

The heat treatment described herein was then carried out on the supernatant.

‘r’ L supernatant was reduced in volume to ‘s’ L by ultrafiltration using a polyvinylidene flouride (PVDF) membrane having a molecular weight cut-off of ‘t’ daltons. The concentrate contained ‘u’ % protein by weight. With the additional protein recovered from the supernatant, the overall protein recovery of the filtered protein solution was ‘v’ wt %. The concentrate was then heated to 85° C. for 10 minutes before being subjected to a further centrifugation step for clarification. The resulting ‘w’ L of centrate having a protein content of ‘x’ % by weight was subjected to a colour reduction step by passing it through a ‘y’ L BV of adsorbent resin at a rate of ‘z’ BV/hr. The ‘aa’ L of resin treated solution having reduced colour and a protein content of ‘ab’ % by weight was then spray dried, given designation ‘s’ C200HR and had a protein content of ‘ac’ wt % (N×6.25) d.b.

The parameters ‘a’ to ‘ac’ for Examples 1 and 2 are set forth in the following Table I:

TABLE I

| | Example 1 | Example 2 |
|---|---|---|
| q | BW-SD076-I24-07A | BW-SD062-A12-06A |
| a | 170 | 98 |
| b | 1,700 | 1,080 |
| c | 0.15 | 0.15 |
| d | 1,321.5 | 735.8 |
| e | 1.55 | 1.50 |
| f | 1,280 | 1,020 |
| g | 1.55 | 1.37 |
| h | 1280 | 1,020 |
| i | 88.35 | 37.5 |
| j | 100,000 | 30,000 |
| k | 19.24 | 14.35 |
| l | 30 | 29 |
| m | 1:15 | 1:10 |
| n | 4 | 4 |
| o | 57 | 17 |
| p | 98.99 | 102.21 |
| r | 1,346 | 398 |
| s | 62.4 | 30.2 |
| t | 5,000 | 5,000/30,000 |
| u | 240 | 6.22 |
| v | 8.43 | 30.7 |
| w | 84 | 22.3 |
| x | 58.1 | 4.40 |
| y | 5 | 5 |
| z | 2 | 2 |
| aa | 58.8 | 25.3 |
| ab | 7.94 | 2.64 |
| ac | 88.98 | 70.76 |

### Example 3

This Example illustrates the production of a novel canola protein product of less than 90% protein by weight in accordance with another embodiment of the invention.

‘a’ kg of canola meal was added to ‘b’ L of ‘c’ M NaCl solution at ambient temperature and agitated for 30 minutes to provide an aqueous protein solution. The residual canola meal was removed and the resulting protein solution was partially clarified by centrifugation to produce ‘d’ L of partially clarified protein solution having a protein content of ‘e’ % by weight. The partially clarified protein solution was then filtered to further clarify, resulting in a solution to volume ‘f’ having a protein content of ‘g’ by weight.

A ‘h’ L aliquot of the protein extract solution was reduced in volume to ‘i’ L by concentration on a polyethersulfone (PES) membrane having a molecular weight cut-off ‘j’ daltons. The retentate was then pasteurized at 60° C. for 1 minute. The resulting pasteurized concentrated protein solution had a protein content of ‘k’ % by weight.

The concentrated solution at ‘l’ ° C. was diluted ‘m’ into cold RO water having a temperature ‘n’ ° C. A white cloud formed immediately and was allowed to settle. The upper diluting water was removed and the precipitated, viscous, sticky mass (PMM) was recovered by centrifugation in a yield of ‘o’ wt % of the filtered protein solution and spray dried. The dried PMM derived protein was found to have a protein content of ‘p’ wt % (N×6.25) d.b. The product was given a designation ‘q’ C300.

The iso-electric precipitation step described herein was then carried out on the supernatant.

‘r’ L supernatant was adjusted to a conductivity of approximately ‘s’ ms by the addition of sodium chloride. The resulting solution was then acidified to a pH of T by the addition of HcL which resulted in the precipitation of 7S protein. The acidified solution was then clarified by centrifugation and filtration to provide a ‘u’ L solution having a protein content of ‘v’. The clarified protein solution was then concentrated by ultrafiltration using polyethersulfone (PES) membranes having a molecular weight cut-off of ‘w’ daltons. The concentrated solution was then diafiltered with a volume of ‘x’ L of pH 3 RO water. The diafiltered solution contained ‘y’ % protein by weight. With the additional protein recovered from the supernatant, the overall protein recovery of the filtered protein solution was ‘z’ wt %. A ‘aa’ L aliquot of retentate was subjected to a colour reduction step by passing it through an ‘ab’ L BV of granular activated carbon at a rate ‘ac’ BV/hr. The ‘ad’ L of carbon treated solution having reduced colour and a protein content of ‘ae’ % by weight was then polish filtered and spray dried, given designation ‘q’ C200ISC and had a protein content of wt % (N×6.25) d.b. The parameters ‘a’ to ‘af’ for Example 3 are set forth in the following Table II:

TABLE II

| | Example 3 |
|---|---|
| q | BW-SD087-L03-07A |
| a | 60 |
| b | 600 |
| c | 0.15 |
| d | 492 |
| e | 1.65 |
| f | 446 |
| g | 1.48 |
| h | 452 |
| i | 27.1 |
| j | 100,000 |
| k | 16.59 |
| l | 31 |
| m | 1:15 |
| n | 3 |
| o | 37 |
| p | 100.41 |
| r | 530 |
| s | 19 |
| t | 3 |
| u | 525 |
| v | 0.24 |
| w | 10,000 |
| x | 120 |
| y | 5.35 |
| z | 58.7 |
| aa | 17.3 |
| ab | 1.7 |
| ac | 2.5 |
| ad | 18.1 |
| ae | 5.10 |
| af | 88.62 |

### Example 4

This Example shows the colour and clarity of liquid samples at pH 3 for the supernatant derived canola protein products produced in Examples 1, 2 and 3.

The dried canola protein samples BW-SD076-I24-07A, BW-SD062-A12-06A and BW-SD087-L03-07A, produced respectively in Examples 1, 2 and 3, along with a dried canola protein isolate sample BW-SD062-A12-06A, were made up into aqueous solutions with a protein content of 3.2 w/v % at their natural pH. The solutions were mixed until fully solubilized and then analyzed on a HunterLab ColorQuest XE instrument for colour and clarity. The results obtained are set forth in the following Table III:

TABLE III

| | Dry | | | | | | |
|---|---|---|---|---|---|---|---|
| | Protein | Solution | Solution | Colour Analysis | | | Haze |
| | Content | pH | Protein | L* | a* | b* | (%) |
| Example 1 | 88.98 | 2.96 | 3.2% | 91.46 | −2.85 | 30.15 | 0% |
| BW-SD076-I24-07A | | | | | | | |
| Example 2 | 70.76 | 3.03 | 3.2% | 91.78 | −2.47 | 26.64 | 2.2% |
| BW-SD062-A12-06A | | | | | | | |
| Example 3 | 88.62 | 3.26 | 3.2% | 92.49 | −2.68 | 26.47 | 5.1% |
| BW-SD087-L03-07A | | | | | | | |
| Canola Protein Isolate | 96.6% | 3.45 | 3.2% | 90.82 | −3.48 | 29.88 | 2.9% |
| BW-SA081-C03-08 | | | | | | | |

As may be seen from Table III, when compared to a canola protein isolate, colour and clarity values for products produced in Example 1, Example 2 and Example 3 are very similar. The three canola protein products have higher L* values than the canola protein isolate, which would indicate a whiter colour. The a* value readings for the two canola protein products are evidence of solutions that are less red in colour than the isolate while the b* values indicate one solution being slightly more yellow while one is slightly less yellow than the isolate. Haze values are very low for all of the products, which shows that they are all very clear when solubilized.

These colour and clarity observations make it possible to conclude that the canola protein products produced in Examples 1, 2 and 3, having less than 90 wt % protein (N×6.25) d.b., are very comparable to a canola protein isolate and are suitable for use in similar food and beverage applications to those of the canola protein isolate.

### SUMMARY OF THE DISCLOSURE

In summary of this disclosure, 2S-predominated canola protein products are produced of comparable properties in aqueous solutions to the 2S-predominated canola protein isolates produced in U.S. Pat. Nos. 11/038,086 and 12/213,500. Modifications are possible within the scope of the invention.

What we claim is:

1. A canola protein product having a protein content of less than about 90 wt % (N×6.25) on a dry weight basis (d.b.) and containing at least about 85 wt % of 2S canola protein and less than about 15 wt % of 7S canola protein of the canola protein present in the isolate.

2. The canola protein product of claim 1 wherein the isolate contains at least about 90 wt % of 2S canola protein and less than about 10 wt % of 7S canola protein of the canola proteins present in the isolate.

3. The canola protein product of claim 1 having a protein content of at least about 60 wt % (N×6.25) d.b.

4. The canola protein product of claim 1 which is obtained by heat treatment of aqueous supernatant, partially concentrated supernatant or fully concentrated supernatant from canola protein micelle formation, removal of precipitate and drying the residual solution.

5. The canola protein product of claim 1 which is obtained by isoelectric precipitation of aqueous supernatant, partially concentrated supernatant or fully concentrated supernatant from canola protein micelle formation, removal of precipitate and drying the residual solution.

6. A process for the preparation of a canola protein product having an increased proportion of 2S canola protein, which comprises:
(a) providing an aqueous solution of 2S and 7S proteins consisting predominantly of 2S protein,
(b) heat treating the aqueous solution to cause precipitation of 7S canola protein,
(c) removing degraded 7S protein from the aqueous solution, and
(d) recovering a canola protein product having a protein content of less than about 90 wt % (N×6.25) d.b. and having an increased proportion of 2S canola protein.

7. The process of claim 6 wherein said heat treatment step is effected under temperature and time conditions sufficient to degrade at least about 50 wt % of the 7S canola protein present in said aqueous solution.

8. The process of claim 7 wherein said heat treatment step degrades the 7S canola protein by at least 75% of 7S canola protein present in said aqueous solution.

9. The process of claim 6 wherein said heat treatment step is effected by heating the aqueous solution for about 5 to about 15 minutes at a temperature of about 75° to about 95° C.

10. The process of claim 6 wherein said aqueous solution of 2S and 7S canola proteins is supernatant, partially concentrated supernatant or concentrated supernatant from canola protein micelle formation and precipitation.

11. The process of claim 10 wherein said canola protein micelle formation is effected by:
(a) extracting canola oil seed meal at a temperature of at least about 5° C. to cause solubilization of protein in said canola oil seed meal and to form an aqueous protein solution,
(b) separating said aqueous protein solution from residual oil seed meal,
(c) increasing the concentration of said aqueous protein solution to at least about 200 g/L while maintaining the ionic strength substantially constant by a selective membrane technique to provide a concentrated protein solution,
(d) diluting said concentrated protein solution into chilled water having a temperature of below about 15° C. to cause the formation of the protein micelles, and
(e) separating supernatant from settled protein micellar mass.

12. The process of claim 11 wherein said supernatant is concentrated to a protein concentration of about 100 to about 400 g/L prior to said heat treatment.

13. The process of claim 12 wherein said supernatant is concentrated to a protein concentration of about 200 to about 300 g/L.

14. The process of claim 12 wherein said concentration step is effected by ultrafiltration using membrane having a molecular weight cut-off about 3,000 to about 100,000 daltons.

15. The process of claim 14 wherein the concentrated supernatant resulting from ultrafiltration is subjected to diafiltration prior to said heat treatment step.

16. The process of claim 15 wherein said diafiltration step is effected using from about 2 to about 20 volumes, preferably about 5 to about 10 volumes, of water using a membrane having a molecular weight cut-off of about 3,000 to about 100,000 daltons.

17. The process of claim 6 wherein said canola protein isolate has a protein content of at least about 60 wt % (N×6.25) d.b.

18. A process for the preparation of a canola protein product having an increased proportion of 2S canola protein, which comprises:
(a) providing an aqueous solution of 2S and 7S proteins consisting predominantly of 2S protein,
(b) isoelectrically precipitating 7S protein from the aqueous solution,
(c) removing precipitated 7S protein from the aqueous solution, and,
(d) recovering a canola protein product having a protein content of less than about 90 wt % (N x 6.25) d.b. and having an increased proportion of 2S canola protein compared to the aqueous solution of 2S and 7S proteins.

19. The process of claim 18 wherein said isoelectric precipitation is effected under pH and salt conditions sufficient to precipitate at least about 50 wt % of the 7S canola protein present in the aqueous solution.

20. The process of claim 19 wherein said isoelectric precipitation is effected under pH and salt conditions sufficient to precipitate at least about 75 wt % of the 7S canola protein present in the aqueous solution.

21. The process of claim 18 wherein said isoelectric precipitation is effected by:
(i) salinating the aqueous solution to a conductivity of at least about 0.3 mS, and
(ii) adjusting the pH of the salinated aqueous solution to a value of about 2.0 to about 4.0.

22. The process of claim 21 wherein said conductivity is about 10 to about 20 mS and said pH is about 3.0 to about 3.5.

23. The process of claim 18 wherein said aqueous solution of 2S and 7S canola proteins is supernatant, partially concentrated supernatant or concentrated supernatant from canola protein micelle formation and precipitation.

24. The process of claim 23 wherein said canola protein micelle formation is effected by:
(a) extracting canola oil seed meal at a temperature of at least about 5° C. to cause solubilization of protein in said canola oil seed meal and to form an aqueous protein solution,
(b) separating said aqueous protein solution from residual oil seed meal,
(c) increasing the concentration of said aqueous protein solution to at least about 200 g/L while maintaining the ionic strength substantially constant by a selective membrane technique to provide a concentrated protein solution,
(d) diluting said concentrated protein solution into chilled water having a temperature of below about 15° C. to cause the formation of the protein micelles, and
(e) separating supernatant from settled protein micellar mass.

25. The process of claim 24 wherein said supernatant is concentrated to a protein concentration of about 100 to about 300 g/L prior to said isoelectric precipitation.

26. The process of claim 25 wherein said supernatant is concentrated to a protein concentration of about 200 to about 300 g/L.

27. The process of claim 25 wherein said concentration step is effected by ultrafiltration using at least one membrane having a molecular weight cut-off of about 3,000 to about 100,000 daltons.

28. The process of claim 27 wherein the concentrated supernatant resulting from ultrafiltration is subjected to diafiltration prior to said isoelectric precipitation.

29. The process of claim 16 wherein said diafiltration step is effected using from about 2 to about 20 volumes, preferably about 5 to about 10 volumes, of water, saline or acidified water using at least one membrane having a molecular weight cut-off of about 3,000 to about 100,000 daltons.

30. The process of claim 6 or 18 further comprising:
(e) formulating said canola protein product as an aqueous beverage composition.

31. An aqueous solution of the canola protein product of claim 1.

32. The aqueous solution of claim 31 which is a canola protein product fortified beverage.
